(19) United States
(12) Patent Application Publication
(10) Pub. No.: US 2013/0137989 A1
(43) Pub. Date: May 30, 2013

(54) ULTRASOUND PROBE AND CORRESPONDING ULTRASOUND DETECTION SYSTEM

(71) Applicant: GE Medical Systems Global Technology Compan, Waukesha, WI (US)

(72) Inventors: You CHEN, JiangSu (CN); Wenting XIA, JiangSu (CN); Rujun TIAN, JiangSu (CN); Houbing LIU, JiangSu (CN)

(73) Assignee: GE Medical Systems Global Technology Company, LLC, Waukesha, WI (US)

(21) Appl. No.: 13/690,451

(22) Filed: Nov. 30, 2012

(30) Foreign Application Priority Data
Nov. 30, 2011 (CN) 201110405502.9

(51) Int. Cl. A61B 8/00

(52) U.S. Cl. CPC A61B 8/4444
USPC 600/459

(57) ABSTRACT

An ultrasound probe and a corresponding ultrasound detection system comprising at least one button, the function of which can be defined by a user based on different applications. According to a clinical application mode in use, functions of button clicking can be automatically redefined and changed to satisfy needs of more complicated diagnostic conditions. Embodiments of the present invention enable a user to configure the function of a button based on different applications, and provide more flexibility and convenience for a user.

### CROSS-REFERENCE TO RELATED APPLICATIONS

This application claims foreign priority benefits under 35 U.S.C. §119 to co-pending Chinese Patent Application No. 201110405502.9, filed Nov. 30, 2011, which is hereby incorporated by reference in its entirety as part of the present disclosure.

### BACKGROUND OF THE INVENTION

Embodiments of the present invention generally relate to the field of ultrasound detection and, more particularly, to an ultrasound probe and a corresponding ultrasound detection system.

Ultrasound detection is a customary detection means in the modern medical field. The existing ultrasound medical equipment consist of an ultrasound mainframe (or “console”) and a probe.

FIG. 1 is a schematic diagram of an ultrasound probe 10 in the prior art. As shown in FIG. 1, the ultrasound probe 10 in the prior art only comprises one button 11. Moreover, the function of the button 11 is fixed, i.e., it has already been configured before shipment.

In the traditional application field of ultrasound scanning, a user usually uses an operation panel (such as keyboard, trackball or touch panel) to control a user interface, thereby controlling an ultrasound scanning application flow, for example, switching the B Mode (Black and White Mode) to CF Mode (Color Flow Mode), function of adjusting the emission frequency, or freezing and unfreezing, etc.

At present, some novel ultrasound probes have one or two buttons on their heads. These buttons enable a user to control the user interface in another manner, yet the function is quite simple. For example, in a probe system with one button, the button is only defined to have one fixed function, such as freezing or defreezing, and a user can only use the button to perform the fixed action. However, in different applications, a user wants to use the button to perform some other functions, such as printing, saving images, and etc. In addition, in those probe systems with two buttons, the two buttons, in most cases, are defined to perform the functions of button traversal browse and confirmation in all the clinical applications, such as go forward, go backward, enter/ok, cancel, and etc. This simple solution is not suitable for some complicated conditions. The default selected key is at the top left corner of the user interface. If a user wants to change the default selected key to one's desired key, such as “proceed downward” or “proceed upward”, the user should single click or double click the button over and over again to carry out traversal navigation one by one to move selection key by key till the selection reaches the user's target key. Such an application is neither user-friendly nor convenient.

Thus, there exist the following problems in the art, i.e., how to bring the functions of a probe button into full play, how to enable a probe button to redefine its click and press function as the user's most desired function automatically according to different clinical application modes.

It is a purpose of the present invention to solve these problems.

### BRIEF SUMMARY OF THE INVENTION

In accordance with an embodiment of the present invention, there is provided an ultrasound probe. The ultrasound probe comprises at least one button, wherein at least one function of the at least one button is defined by a user based on different applications.

In accordance with an embodiment of the present invention, there is provided an ultrasound detection system. The ultrasound detection system comprises an ultrasound probe comprising at least one button, wherein at least one function of the at least one button is defined by a user based on different applications, and a user interface for the user to define the at least one function of the at least one button, and for making interactive responses.

### BRIEF DESCRIPTION OF THE DRAWINGS

The above and other purposes and characteristics of the present invention would become obvious through the following detailed description made with reference to the figures, where like reference numbers are used to denote the same or similar parts, wherein:

FIG. 1 is a schematic diagram of an ultrasound probe in the prior art;

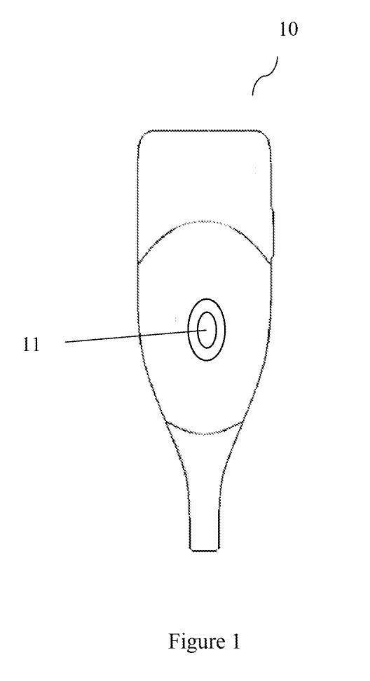

FIG. 2 is a schematic diagram of an ultrasound probe system according to an embodiment of the present invention;

FIG. 3 is a schematic diagram of the operational principle of an ultrasound probe system according to an embodiment of the present invention; and

FIG. 4 is a flow chart of a method for using an ultrasound probe system according to an embodiment of the present invention.

### DETAILED DESCRIPTION OF THE INVENTION

One or more embodiments of the present invention provide an ultrasound probe comprising at least one button, the function of which can be defined by a user based on different applications.

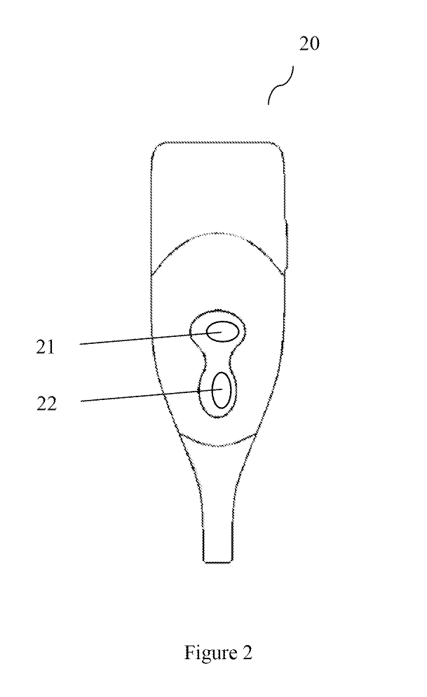

FIG. 2 is a schematic diagram of an ultrasound probe system according to an embodiment of the present invention. As shown in FIG. 2, an ultrasound probe 20 may comprises two buttons 21 and 22.

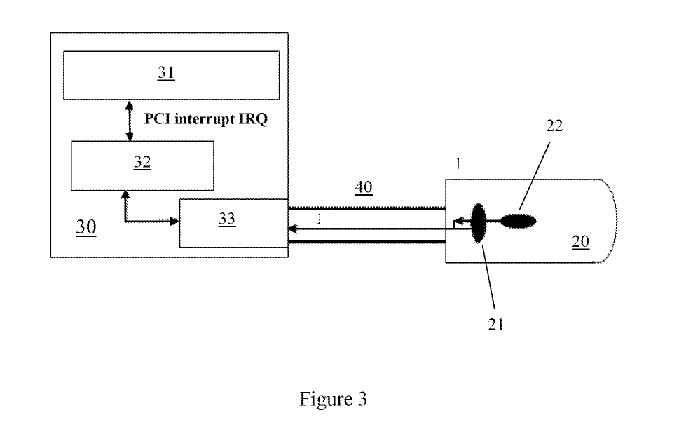

FIG. 3 is a schematic diagram of the operational principle of an ultrasound probe system according to an embodiment of the present invention. A button signal of probe buttons 21 and 22 is transmitted through a probe cable 40 to a probe connector 33 in an ultrasound mainframe 30, as shown by the arrow connecting the probe buttons 21 and 22 to the probe connector 33. The button signal is then input to a button signal decoding circuit 32. The button signal decoding circuit 32 decodes the button signal and outputs PCI or ePCI bus interrupt request (IRQ) to an interactive application user interface 31. After receiving the interrupt request from the PCI bus, the user interface 31 presents a corresponding display and makes an interactive response. Thus, the user can control the user interface 31 by operating a mouse or the probe button 21 or 22, thereby defining the functions of the probe button 21 or 22.

A surgical operation can be used, for example, to explain FIGS. 2 and 3. Before the operation, the probe needs to be disinfected, and a surgeon needs to wear surgical gloves. However, it is impossible to disinfect the ultrasound mainframe 30. Thus, a special cover or slipcover is usually used to cover the ultrasound mainframe 30. In the process of the operation, the surgeon sometimes needs to save images. In the prior art, images are saved by operating the ultrasound mainframe 30. Since the ultrasound mainframe is covered and the surgeon's view is affected, it is quite inconvenient to operate. According to embodiments of the present invention, the surgeon can define the functions of the button 21 or 22 in this case to “save images” in advance. In the process of the operation, the surgeon only needs to press the corresponding button 21 or 22 to save images conveniently. In addition, for the convenience of manipulations in the operation, the two buttons 21 and 22 can be manufactured in different orientations, as shown in FIG. 2, so that the surgeon can easily distinguish the two buttons from each other merely by touch. The two buttons 21 and 22 can also be manufactured in different shapes, or in other manners occurred to persons skilled in the art which make it easy to distinguish the button 21 from the button 22.

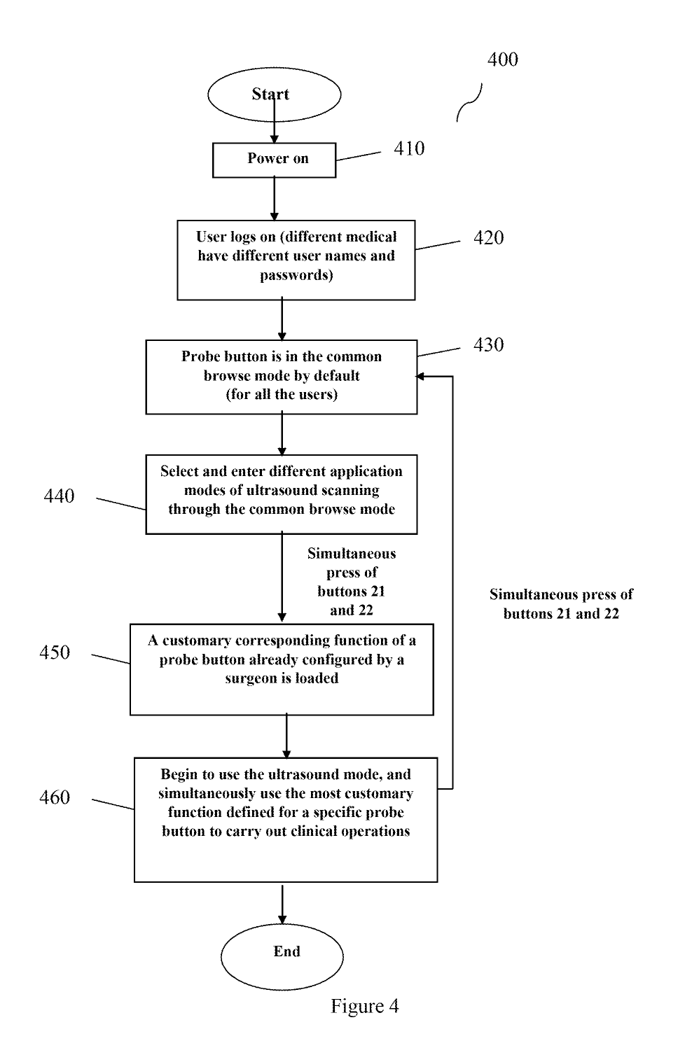

A NICU (Neonatal Intensive Care Unit) probe can be used, for example, to explain the operational principle of a probe according to an embodiment of the present invention with reference to FIG. 4. FIG. 4 is a flow chart of a method for using an ultrasound probe system according to an embodiment of the present invention. In general cases, the probe 20 may comprise two buttons; the two buttons are defined to perform the general function of browsing the user interface through button pressing; single click of the button 21 is “go forward”, double click of the same button “go backward”, single click of the button 22 “enter/ok”, and double click of the same button “cancel”.

A method 400 begins with turning the power of the machine on 410. In an embodiment, a user powers the machine on.

A user logs on 420. In an embodiment, different surgeons have different user names and passwords.

After logging on, the probe button is in common browse mode (for all the users) by default 430. At this point, the user interface displays the default home page. The user can change or adjust the definitions of the most customary functions of the two buttons on the probe in different ultrasound scanning application modes. In addition, from here, the user can use the buttons 21 and 22 to traverse operations one by one, and select and enter a target clinical application page or predetermined page. In an embodiment, different ultrasound scanning application modes are selected to enter through the common browse mode 440.

After entering a specific application, pressing the buttons 21 and 22 simultaneously redefines the default function of the buttons 21 and 22 as the user's most desired function instead of the common function of browsing the user interface through button pressing. In an embodiment, a customary corresponding function of a probe button already configured by a surgeon is loaded according to a user name 450. The ultrasound mode is initiated 460; meanwhile, the most customary function defined for a specific probe button is used to carry out clinical operations. For example, if a user enters “superficial quick scanning” mode, single click of the button 21 would be changed from “go forward” to “gain increase”, and single click of the button 22 from “enter/ok” to “gain decrease”. Thus, as long as the user enters this application mode, the user can use the probe buttons to adjust the gain level directly, without pressing “go forward” again and again to navigate the select key to gain adjusting function key. This is very convenient to the user.

After adjusting the gain to the proper level, the user only needs to single click the button 21 and the button 22 (the cancel function) simultaneously to return to standard default functions of the buttons 21 and 22, i.e., “go forward”, “go backward”, and “enter/ok” functions in other common modes, as shown by the arrow from block 460 to block 430 in FIG. 4.

For some other applications, such as CF mode, simultaneous single click of the buttons 21 and 22 can be redefined as “frequency increase” and “frequency decrease”. Likewise, after obtaining the desired frequency, single clicks the buttons 21 and 22 simultaneously to exit the redefinition mode, and return to the standard function of browsing the user interface through button pressing.

For those non-clinical applications, such as “patient management”, single click of the button 21 can be redefined as “next patient”, single click of the button 22 “return”, double click of the button 21 “output”, and double click of the button 22 can return to the function of browsing the user interface through button pressing.

In some embodiments, two or more buttons can be integrated together so that they look like a single button in appearance.

According to an embodiment of the present invention, for a probe that has only one button, the function of the button can also be redefined based on different application modes. For example, when entering B Mode, the button is redefined to perform the freezing/defreezing function, double click of the button can also be redefined as “gain increase”, single click “gain decrease”, and long press is redefined to return to the standard operations.

For the purpose of more flexibility in user's selections, there may be a predetermined page available to a user on the user interface so that the user can personalize the configuration of different clicks of these buttons to perform the user's most desired function. As a result, the user can use the probe buttons most effectively.

It should be noted that FIG. 4 is only exemplary. According to different embodiments of the present invention, the method for using the ultrasound probe system may comprise more or less different steps.

Table 1 shows examples of the most customary functions defined by default of the buttons 21 and 22 of the ultrasound probe 20 according to an embodiment of the present invention.

TABLE 1

| The most customary | B Mode | |
|---|---|---|
| functions defined by | (Black and White | CF Mode (Color Flow |
| default | Ultrasound Mode) | Ultrasound Mode) |
| Single click of button 21 | brightness (gain) | color gain increase |
| | increase | |
| Single click of button 22 | brightness (gain) | color gain decrease |
| | decrease | |
| Long press of button 21 | brightness (gain) | color gain increase on |
| | increase on | |
| Long press of button 22 | brightness (gain) | color gain decrease on |
| | decrease on | |
| Double click of button | scanning depth | PRF (Pulse Repetition |
| 21 | increase | Frequency) increase |
| Double click of button | scanning depth | PRF (Pulse Repetition |
| 22 | decrease | Frequency) decrease |
| Simultaneous press of | exit the customary | exit the customary |
| buttons 21 and 22 | definition mode, | definition mode, |
| | return to the | return to the |
| | common browse | common browse |
| | mode | mode |

Table 2 shows examples of functions of buttons 21 and 22 in the common browse mode.

TABLE 2

| | |
|---|---|
| The common browse | same in some of specific ultrasound application |
| mode | modes |
| Single click of button 21 | go forward: move to the next application key |
| Single click of button 22 | go backward: move to the last application key |
| Long press of button 21 | move on (forward) |
| Long press of button 22 | move on (backward) |
| Double click of button 21 | confirm and enter |
| Double click of button 22 | return to higher level |
| Simultaneous press of | return to the customary definition mode |
| buttons 21 and 22 | |

It should be noted that Table 1 and Table 2 are exemplary rather than restrictive. In practice, manufacturers and users can design different button functions based on different applications.

An embodiment of the present invention further provides an ultrasound detection system comprising the ultrasound probe according to an embodiment of the present invention and a user interface, wherein the user interface is for a user to define the function of the at least one button and for making interactive responses.

The ultrasound probe according to the an embodiment of the present invention can automatically redefine and change functions of button clicking based on a clinical application mode in use to satisfy needs of more complicated diagnostic conditions. The user interface according to embodiments the present invention enables the user to configure functions of a button based on different applications, thereby providing more flexibility and convenience for the user.

Although the NICU probe is described as an example above, it should be understood that the present invention is not limited to the NICU probe but can be applied to all the ultrasound probes.

It should be noted that the above embodiments are merely exemplary rather than restrictive. Without deviating from the scope of the attached claims, ones skilled in the art could design a great number of alternative embodiments. The adopted verb “comprise” does not exclude elements and steps other than those recorded in the claims or description. Prior to element, the expression “a/an” does not exclude the existence of more than one element of such. In the claims, any bracketed reference numbers should not be understood as limitations to the claims.

What is claimed is:

1. An ultrasound probe, comprising:
at least one button, wherein at least one function of the at least one button is defined by a user based on different applications.

2. The ultrasound probe according to claim 1, wherein the at least one button comprises two or more buttons.

3. The ultrasound probe according to claim 2, wherein the two or more buttons are integrated together.

4. The ultrasound probe according to claim 2, wherein the two or more buttons vary in directions or shapes.

5. The ultrasound probe according to claim 1, wherein the at least one function of the at least one button comprises functions associated with single clicking, double clicking, or long pressing of the at least one button, or simultaneously pressing of more than one buttons of the at least one button.

6. The ultrasound probe according to claim 1, wherein the at least one function of the at least one button comprises functions of adjusting scanning parameters or image parameters.

7. The ultrasound probe according to claim 1, wherein the at least one function of the at least one button comprises a function of browsing a user interface buttons.

8. The ultrasound probe according to claim 1, wherein the at least one function of the at least one button comprises a function of switching different button operation modes.

9. An ultrasound detection system, comprising:
an ultrasound probe comprising at least one button, wherein at least one function of the at least one button is defined by a user based on different applications; and
a user interface for the user to define the at least one function of the at least one button, and for making interactive responses.
